(19) United States
(12) Patent Application Publication
(10) Pub. No.: US 2022/0332586 A1
(43) Pub. Date: Oct. 20, 2022

(54) PREPARATION AND APPLICATION OF 2,6-DIAMINOANTHRAQUINONE BIFUNCTIONAL GROUP COVALENTLY GRAFTED GRAPHENE AS NEGATIVE MATERIAL OF SUPERCAPACITOR

(71) Applicant: GUANGDONG UNIVERSITY OF TECHNOLOGY, GUANGZHOU (CN)

(72) Inventors: Miao HE, GUANGZHOU (CN); Yandong XIE, GUANGZHOU (CN); Yefeng FENG, GUANGZHOU (CN); Deping XIONG, GUANGZHOU (CN)

(21) Appl. No.: 17/229,983

(22) Filed: Apr. 14, 2021

(51) Int. Cl. C01B 32/194; C01B 32/192; C07C 221/00; H01G 11/42

(52) U.S. Cl. CPC C01B 32/194; C01B 32/192; C07C 221/00; H01G 11/42; C01B 2204/22; C01P 2004/03; C01P 2004/04; C01P 2002/82

(57) ABSTRACT

The present invention discloses a preparation method of 2,6-diaminoanthraquinone bifunctional covalently grafted graphene as a negative material of a supercapacitor, which includes: first dispersing graphite oxide in deionized water; after stirring and ultrasonic treatment, reducing the graphite oxide into reduced graphene oxide by using a hydrazine hydrate, and vacuum drying at 40-80° C.; dispersing the reduced graphene oxide in a DMF solution with 2,6-diaminoanthraquinone, and stirring and performing the ultrasonic treatment again; at 60-90° C., adding isoamyl nitrite, and reacting for 18-24 h; and washing reaction products with ethanol and deionized water for multiple times, and finally freeze drying to obtain a product.

### TECHNICAL FIELD

The present invention relates to preparation of a graphene-based covalently grafted composite material, and particularly to preparation of a 2,6-diaminoanthraquinone functional graphene three-dimensional network structure composite material. The present invention further relates to an application of the 2,6-diaminoanthraquinone functional graphene three-dimensional network structure composite material as an electrode material in a supercapacitor, and belongs to the technical fields of composite materials and supercapacitors.

### BACKGROUND OF THE PRESENT INVENTION

With daily depletion of the fossil energy and rising prices of crude oil and coal in the world, the energy problem has become increasingly prominent. Furthermore, in the application process, the fossil energy always has the problems of high pollution and high energy consumption. Moreover, the fossil energy is also a nonrenewable primary energy. Therefore, the energy problem has become one of main human survival and development problems that have to be solved. With the increasing popularization of renewable energy such as solar energy, wind energy, and hydroenergy, an effect of alleviating the energy shortage has been achieved. However, these renewable secondary energy sources cannot be used on large scale due to the restriction of natural factors such as geographical areas. Therefore, the energy storage problem becomes more acute.

As an important energy storage and energy conversion apparatus, the supercapacitor has the characteristics of fast power output, strong reversibility, low maintenance cost, high charge and discharge speed, and long cycle life, and plays an important role in supplementing batteries in various fields. Compared with the traditional capacitor, the supercapacitor has energy density and power density higher by several orders of magnitude and is wider in application prospects. However, compared with the traditional battery, the energy storage is apparently lower. Therefore, a feasible method is urgently needed to improve the energy density of the supercapacitor. An electrode material is a main factor that determines the performance of the capacitor. The conventional types of the electrode material are as follows: carbon-based materials, metal (hydrogen) oxides, conductive polymers, and small organic molecules with pseudocapacitive properties. The small organic molecules with pseudocapacitance properties have electrochemically-active functional groups, and are high in theoretical capacitance. With abundant raw materials, the small organic molecules with pseudocapacitance properties are green and renewable energy sources, which can chemically modify the carbon-based materials. Secondly, in the energy storage process, the small organic molecules with pseudocapacitive properties have a consecutive reversible redox process only at the oxygen-containing functional groups above, and an intrinsic carbon skeleton may not be destroyed, which provides an important guarantee for obtaining long-term cycle stability. Compared with the traditional double-electric-layer carbon material, the small organic molecules with pseudocapacitive properties have redox functional groups with high electrochemical activity. The multi-electron reversible Faraday reaction at low molecular weight can be realized, and then the Faraday reaction occurs, so that the performance of electrochemical capacitors can be improved.

### SUMMARY OF PRESENT UTILITY MODEL

A purpose of the present invention is to provide a preparation method of 2,6-diaminoanthraquinone bifunctional group covalently grafted graphene as a negative material of a supercapacitor.

Another purpose of the present invention is to study electrochemical capacitance performance of the 2,6-diaminoanthraquinone bifunctional group covalently grafted graphene as the negative material of the supercapacitor so as to use the 2,6-diaminoanthraquinone bifunctional group covalently grafted graphene as an electrode material of the supercapacitor.

I. Preparation of 2,6-diaminoanthraquinone bifunctional group covalently grafted graphene as a negative material of a supercapacitor.

The preparation method of the 2,6-diaminoanthraquinone bifunctional group covalently grafted graphene as the negative material of the supercapacitor in the present invention includes the following technological steps:

(1) dispersing 0.1-1.0 g of graphite oxide in deionized water, stirring for 1-2 h in advance, and then performing ultrasonic treatment for 2-6 h; and adding 10-15 ml of hydrazine hydrate at 80-110° C., and vacuum drying solid substances at 40-80° C. to obtain a reduced graphite oxide substrate;

(2) dissolving 0.1-0.7 g of 2,6-diaminoanthraquinone in a DMF solution, stirring for 1-2 h, adding the reduced graphite oxide into the above solution, continuously stirring for 2-4 h, and then performing the ultrasonic treatment for 4-8 h; when the mixed solution is heated to 60-90° C., adding isoamyl nitrite, and reacting for 18-24 h; washing reaction products with ethanol and deionized water for multiple times, and finally freeze drying to obtain a target product;

A mass ratio of 2,6-diaminoanthraquinone to the graphite oxide is 0.1:1-0.4:2.

II. Physical characterization of 2,6-diaminoanthraquinone bifunctional group covalently grafted graphene as the negative material of the supercapacitor

The covalently grafted graphene material prepared in embodiment 2 is taken as an example.

The structure of the 2,6-diaminoanthraquinone covalently grafted graphene material in the present invention is characterized. The morphology of the product is observed by a scanning electron microscope (SEM ULTRA Plus, Germany) and a transmission electron microscope (TEM JEOL, JEM-2010, Japan); an infrared spectrum (FT-IR) is analyzed by a Nicolet Nexus 670 Fourier transform infrared spectrometer; and Brunauer-Emmett-Teller method (BET) and Barrett-Joyner-Halenda method (BJH) are used to respectively analyze the specific surface area and pore size distribution.

### 1. Field emission scanning electron microscope

FIG. 1 is a field emission scanning electron microscope (SEM) image of reduced graphene oxide (RGO) of a substrate used in the present invention. It can be seen from FIG. 1 that reduced graphene oxide sheets are overlapped and combined with each other to form a thick solid layer.

FIG. 2 is a field emission SEM image of a 2,6-diaminoanthraquinone covalently grafted graphene composite material (DAAQ-RGO) prepared in the present invention. It can be seen that after the covalent functionalization of 2,6-diaminoanthraquinone, a wrinkled surface can be observed easily, which indicates that a lamellar structure of the covalently functionalized graphene is separated, so that the graphene can be prevented from closely stacking together due to the effect of Van der Waals' force. This provides richer channels and more active sites for the permeation of electrolyte ions, so that the electrode material can fully contact the electrolyte ions, and the pseudocapacitance properties of the material can also be improved.

### 2. Transmission electron microscope

FIG. 3 is a transmission electron microscope (TEM) image of the 2,6-diaminoanthraquinone covalently grafted graphene composite material (DAAQ-RGO) prepared in the present invention. It is observed that the composite material presents a flexible and wrinkled morphology. Compared with the RGO, the DAAQ-RGO sheets are more wrinkled and shrunk because some DAAQ molecules are connected to two different graphene lamellar molecules at both sides in the functionalization process.

### 3. FI-IR analysis

FIG. 4 shows infrared spectra of pure 2,6-diaminoanthraquinone, RGO, and DAAQ-RGO respectively. It can be seen from FIG. 4 that the organic molecules are successfully covalently grafted onto the graphene surface, and the peak position of the composite material is almost the same as that of an organic matter, and there is no peak of C-N bonds in DAAQ-RGO. The result shows that the covalent grating of DAAQ on the graphene sheets is successful.

### 4. N₂ adsorption and desorption analysis

A microstructure of the DAAQ-RGO electrode material is further proved by nitrogen adsorption and desorption isotherm and pore size distribution curves. As shown in FIG. 5, the curves respectively show characteristics of an IV-type istotherm curve, which shows a mesoporous structure of the DAAQ-RGO composite material. The DAAQ-RGO composite material has apparent loopbacks (P/P₀ is about 0.4-1) under relative pressure, which proves that there are a lot of mesopores. In addition, the specific surface area of a DAAQ-PGN sample measured by the Brunauer-Emmett-Teller(BET) method is 108.4 m² g⁻¹ , which shows that 2,6-diaminoanthraquinone plays an important role in the growth process of graphene toward larger specific surface area and further indicates that the functionalized graphene sheets can effectively inhibit the close face-to-face stacking, thereby forming mesoporous channels of the electrolyte ions.

III. Electrochemical performance of 2,6-diaminoanthraquinone bifunctional group covalently grafted graphene as the negative material of the supercapacitor.

The electrochemical performance characterization of the 2,6-diaminoanthraquinone bifunctional group covalently grafted graphene (DAAQ-RGO) prepared by the present invention is described below in detail through an electrochemical workstation CHI760E.

1. Preparation of a supercapacitor electrode: a solid mixture of DAAQ-RGO composite material and conductive carbon black is weighed in total of 4.7 mg, wherein mass percentages of the DAAQ-RGO and the conductive carbon black are 65%-85% and 35%-15% respectively. Afer uniform mixing, 0.4 mL of 0.25wt% Nafion solution is dropwise added, and ultrasonically dispersed for 3-5 h to form a suspension. 6 μL, of the above suspension is dropwise added to the surface of a glassy carbon electrode, and dried at room temperature for test of electrochemical performance.

### 2. Test of electrochemical performance

A tri-electrode system is formed by taking the above prepared composite material as a working electrode, a conductive carbon rod as a counter electrode and a saturated calomel electrode as a reference electrode. 1 mol L⁻¹ of H₂SO₄ solution is used as an electrolyte solution, and a potential window is set in a range of -0.4 V to 0.6 V.

The electrochemical performance test is carried out on the RGO and the DAAQ-RGO composite material at a scanning rate of 5 mV s⁻¹ in 1 mol L⁻¹ of H₂SO₄ electrolyte solution. The test result is shown in FIG. 6. It can be seen from the figure that the cyclic voltammetry curve of the substrate material RGO presents a rectangular shape, which shows that the energy storage of RGO is mainly a double-electric-layer energy storage mechanism. For the DAAQ-RGO composite electrode material, a pair of apparent redox peaks can be observed at -1.5 V, which is caused by the reversible redox reaction of DAAQ small organic molecules. It can also be observed that double electric layers of the DAAQ-RGO composite material are much larger than those of the substrate material RGO, which indicates that the small organic molecules of DAAQ are successfully covalently grafted to the surface of RGO. The CV curve of the composite material illustrates that the composite material presents the pseudocapacitance properties of the small organic molecules of DAAQ, and the double-electron layer performance of the composite material is also enhanced.

FIG. 7 illustrates a constant-current charge and discharge curve of RGO and DAAQ-RGO composite material at 1 A g⁻¹ in 1 mol L⁻¹ of H₂SO₄ electrolyte solution. It can be seen through comparison from FIG. 7 that the specific capacitance of the DAAQ-RGO composite material is far greater than that of the substrate RGO. The specific capacitance is more than twice of that of the substrate material.

The cyclic voltammetry curves of DAAQ-RGO at different scanning rates are as shown in FIG. 8, and the shapes of all CV curves are almost unchanged, which shows that the material has excellent rate performance and fast current potential response. The good capacitance behavior is caused by the fast ion diffusion behavior of the electrolyte ions on the DAAQ-RGO surface. In addition, the position difference of the oxidization peak and the reduction peak can be neglected, which indicates that the electrochemical reaction of the DAAQ molecules has good dynamic reversibility.

FIG. 9 shows constant-current charge and discharge curves of DAAQ-RGO at different current densities. The non-linear curve of DAAQ-RGO presents a visible voltage platform at low potential. In addition, all non-linear constant-current charge and discharge curves present a voltage platform, which indicates that the charge of DAAQ-RGO is stored through the Faraday reaction. When the current density is 1, 2, 3, 5, 7 and 10 A g⁻¹, the specific capacitance of the materials can be calculated as 412.7, 406.4, 333.6, 316.0, 305.2 and 294.0 F g⁻¹ respectively. When the current density is 10 A g⁻¹, the capacitance is maintained at 71.24% of that at 1 A g⁻¹, which indicates that DAAQ-RGO has excellent rate performance. The outstanding rate performance is related to the fact that the DAAQ molecules are anchored on the RGO surface and have fast reversible Faraday reaction. The unique covalent graft structure provides fast ion dispersion channels and reactive sites for electrolyte ions, and can give full play to the pseudocapacitance performance of the DAAQ small organic molecules.

FIG. 10 shows Nyquist curves of RGO and DAAQ-RGO. The impedance spectrum of DAAQ-RGO at a bias potential of 0.2 V in a frequency range of 0.1 V-10⁵ kHz shows an inconspicuous semicircle at high frequencies (illustration). It can be seen that equivalent series resistances of RGO and DAAQ-RGO are 3.18 Ω and 2.67 Ω respectively. Therefore, compared with RGO, the DAAQ-RGO electrode presents relatively lower resistance. An almost-vertical line in a low frequency region indicates that the capacitance behavior is mainly limited by diffusion. All the results show that DAAQ-RGO has broad prospects in capacitor electrode materials.

In conclusion, the 2,6-diaminoanthraquinone bifunctional group covalently grafted graphene as the negative material of the supercapacitor prepared by the present invention has high specific capacitance and excellent rate performance, and thus can be served as a the negative material of the supercapacitor. In addition, a synthetic route of the reduced graphene oxide covalently grafted by the small organic molecules in the present invention is simple, the operation is convenient, and the cost is low. Moreover, the green and environment-friendly renewable small organic molecules are used as raw materials, so that the large-scale production can be realized, and a new platform is provided for the design and application prospects of the negative material of the supercapacitor.

### DESCRIPTION OF THE DRAWINGS

FIG. 1 is a scanning electron microscope image of reduced graphite oxide (RGO);

FIG. 2 is a scanning electron microscope image of a DAAQ-RGO composite material prepared by the present invention;

FIG. 3 is a transmission electron microscope image of the DAAQ-RGO composite material prepared by the present invention;

FIG. 4 is an FI-IR analysis of RGO, DAAQ and DAAQ-RGO composite electrode material;

FIG. 5 illustrates N2 adsorption and desorption analysis of the DAAQ-RGO composite electrode material;

FIG. 6 illustrates cyclic voltammetry curves of RGO and DAAQ-RGO composite electrode material in 1 mol L⁻¹ of H₂SO₄ electrolyte solution at a scanning rate of 5 mV s⁻¹;

FIG. 7 is a constant-current charge and discharge diagram of RGO and the DAAQ-RGO composite electrode material in 1 mol L⁻¹ of H₂SO₄ electrolyte solution at a current density of 1 A g⁻¹;

FIG. 8 illustrates cyclic voltammetry curves of a DAAQ-RGO composite material electrode in 1 mol L⁻¹ of H₂SO₄ electrolyte solution at different scanning rates;

FIG. 9 illustrates constant-current charge and discharge curves of the DAAQ-RGO composite material electrode in 1 mol L⁻¹ of H₂SO₄ electrolyte solution at different current densities; and

FIG. 10 is an AC impedance diagram of the DAAQ-RGO composite material electrode.

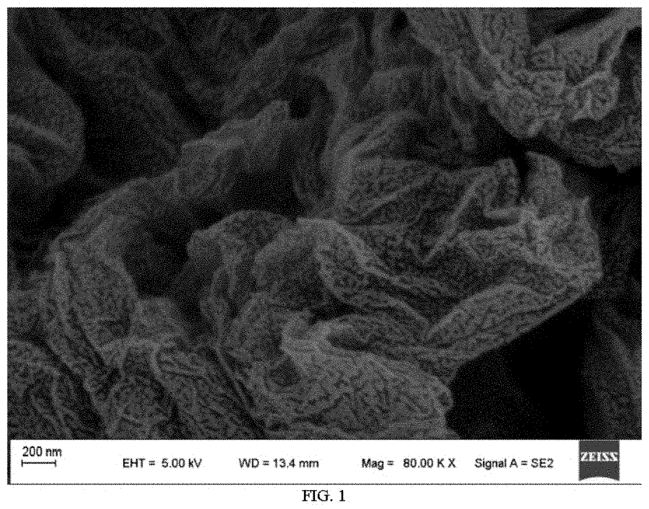

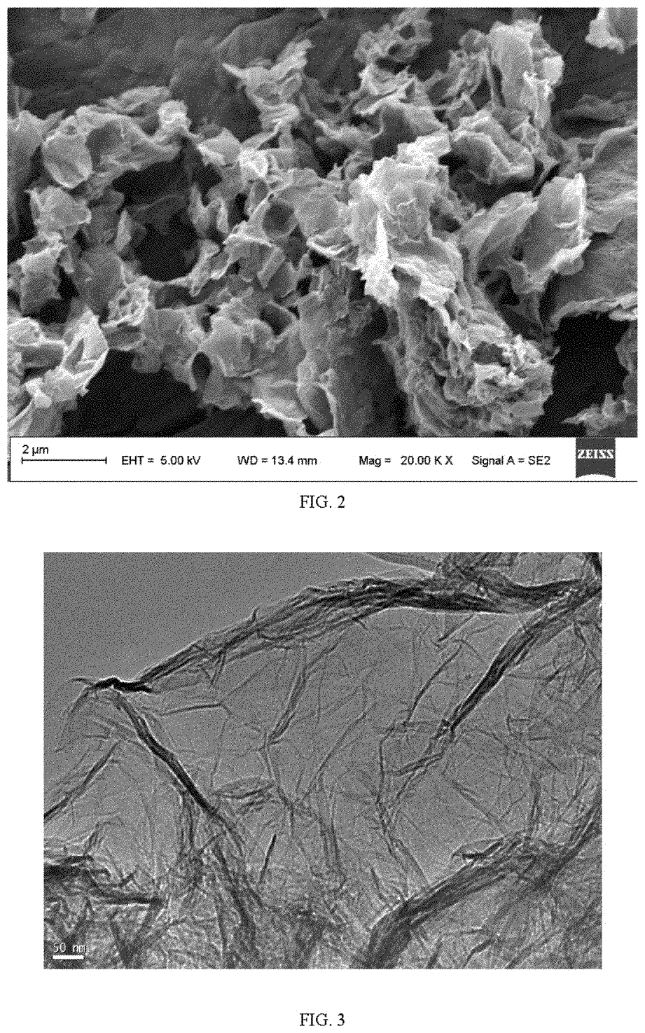

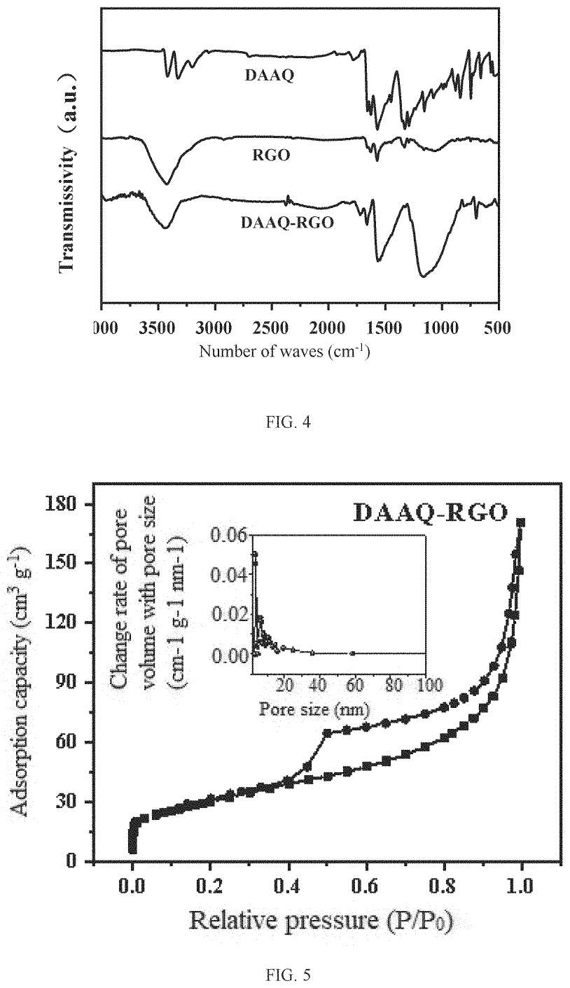

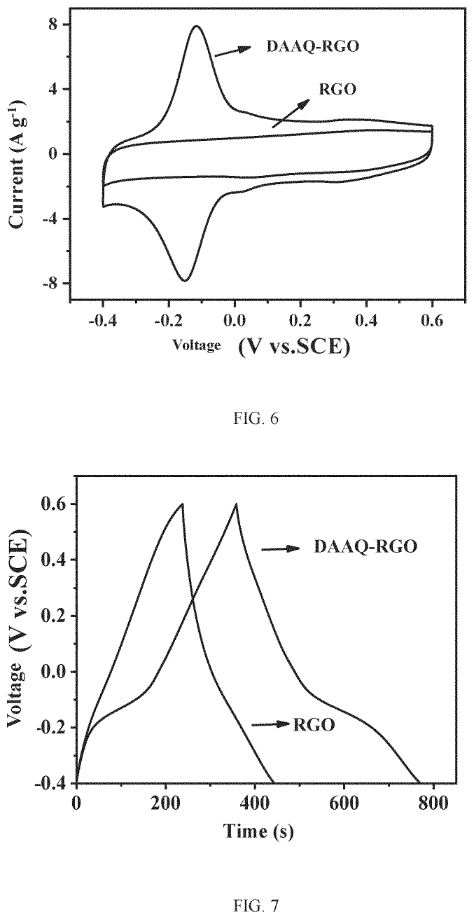

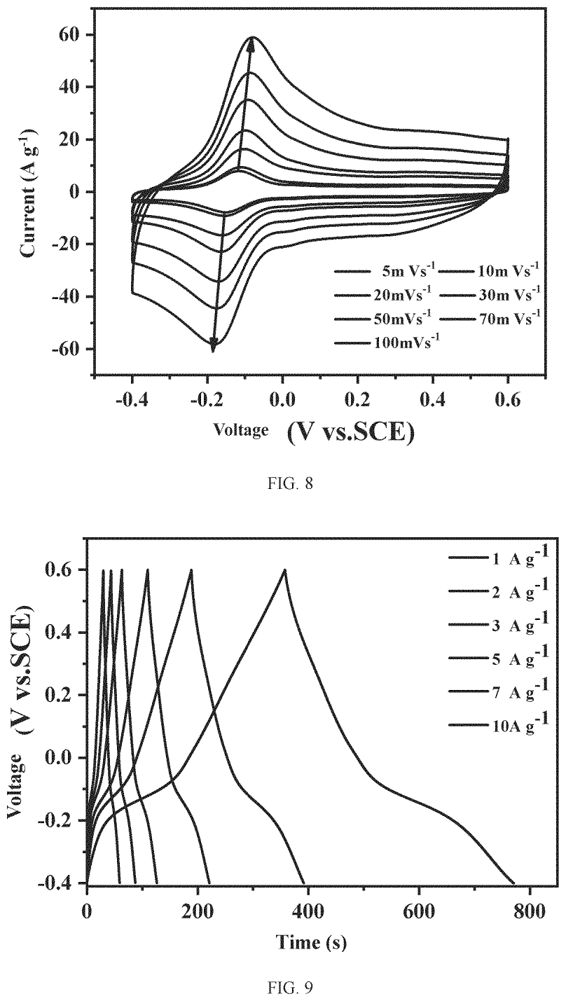

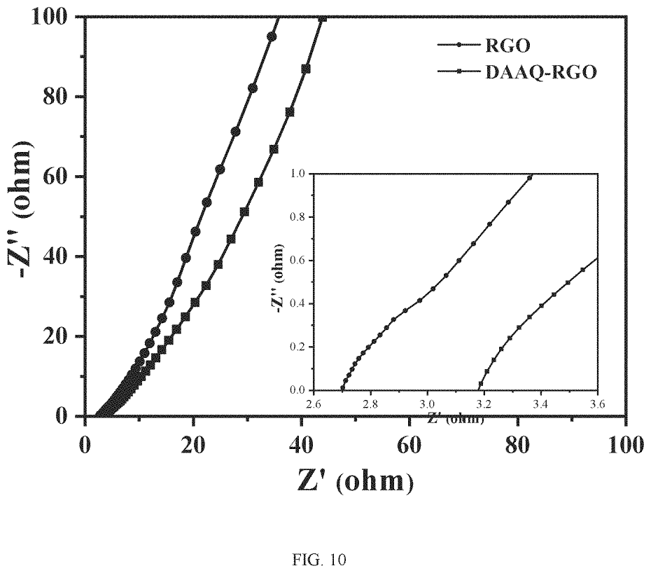

### DETAILED DESCRIPTION OF PREFERRED EMBODIMENTS

Preparation and electrochemical performance of 2,6-diaminoanthraquinone bifunctional group covalently grafted graphene (DAAQ-RGO) as a negative material of a supercapacitor of the present invention is further described in detail below through specific embodiments.

Applied instruments and reagents: CHI760E electrochemical workstation (Shanghai Chenhua Instrument Co., Ltd.) for electrochemical performance test; electronic balance (Beijing Sartorius Instrument Co., Ltd.) for weighing chemicals; transmission electron microscope (TEM JEOL, JEM-2010, Japan); constant-temperature magnetic stirrer (90-1 Shanghai Huxi Analytical Instrument Factory); LGJ-10C freeze drier(Xiangyi Centrifuge Instrument Co., Ltd.); scanning electron microscope (Ultra Plus, Carl Zeiss, Germany) for material morphology characterization; FTS3000 Fourier infrared spectrometer (DIGILAB, America); specific surface area and pore size distribution are tested by a nitrogen adsorption instrument (BET, micromeritics ASAP 2020, America); and 2,6-diaminoanthraquinone (TCI (Shanghai) Chemical Industry Development Co., Ltd.), isoamyl nitrite (Alfa Aesar China Chemical Co., Ltd.), and conductive carbon black (Tansha Graphite Factory in Guiyang, Hunan Province). Water used in the experimental process is deionized water. Reagents used in the experiment are all analytically pure.

### Embodiment 1

### 1. Preparation of a DAAQ-RGO-1 composite material:

(1) 0.1 g of graphite oxide is dispersed in deionized water, stirred for 1 h in advance, and then subjected to ultrasonic treatment for 2 h; and 10 ml of hydrazine hydrate is added at 80° C., and solid substances are vacuum dried at 40° C. to obtain a reduced graphite oxide substrate.

(2) 0.1 g of 2,6-diaminoanthraquinone is dissolved in a DMF solution and stirred for 1 h, then reduced graphite oxide is added into the above solution and stirred continuously for 2 h, and then the ultrasonic treatment is performed for 4 h; when the mixed solution is heated to 60 ° C., isoamyl nitrite is added, and reaction is performed for 18 h; and reaction products are washed with ethanol and deionized water for multiple times and finally freeze dried to obtain a target product.

2. Preparation of a DAAQ-RGO-1 composite material electrode: a solid mixture of DAAQ-RGO composite material and conductive carbon black is weighed in total of 4.7 mg, and the mass percentages of the DAAQ-RGO and the conductive carbon black are 65% and 35% respectively. After the uniform mixing, 0.4 mL of 0.25wt% Nation solution is dropwise added and ultrasonically dispersed for 3 h to form a suspension. 6 of the above suspension is dropwise added to the surface of a glassy carbon electrode and dried at a room temperature for test.

### 3. Test of electrochemical performance:

A tri-electrode system is formed by taking a DAAQ-RGO-1 composite material electrode as a working electrode, a conductive carbon rod as a counter electrode and a saturated calomel electrode as a reference electrode. 1 mol L⁻¹ of H₂SO₄ solution is used as an electrolyte solution, and a potential window is set in a range of -0.4 V to 0.6 V. It can be calculated from the constant-current charge and discharge curve that when the current density is 1 A g⁻¹, the specific capacitance of the electrode material can reach up to 327.4 F g⁻¹.

### Embodiment 2

### 1. Preparation of IT-RGO-2 composite material:

### 1. Preparation of DAAQ-RGO-2 composite material:

(1) 0.1 g of graphite oxide is dispersed in deionized water, stirred for 1 h in advance, and then subjected to ultrasonic treatment for 2 h; and 10 ml of hydrazine hydrate is added at 80° C., and solid substances are vacuum dried at 40° C. to obtain a reduced graphite oxide substrate.

(2) 0.4 g of 2,6-diaminoanthraquinone is dissolved in a DMF solution and stirred for 1 h, then reduced graphite oxide is added into the above solution and stirred continuously for 2 h, and then the ultrasonic treatment is performed for 4 h; when the mixed solution is heated to 60 ° C., isoamyl nitrite is added, and reaction is performed for 18 h; and reaction products are washed with ethanol and deionized water for multiple times and finally freeze dried to obtain a target product.

2. Preparation of the DAAQ-RGO-2 composite material electrode is the same as that in embodiment 1;

3. Test of electrochemical performance: the test method is the same as that in embodiment 1; and the test result is: when the current density is 1 A g⁻¹, the specific capacitance of the electrode material can reach up to 412.7 F g⁻¹.

### Embodiment 3

### 1. Preparation of DAAQ-RGO-3 composite material:

### 1. Preparation of DAAQ-RGO-3 composite material:

(1) 0.1 g of graphite oxide is dispersed in deionized water, stirred for 1 h in advance, and then subjected to ultrasonic treatment for 2 h; and 10 ml of hydrazine hydrate is added at 80° C., and solid substances are vacuum dried at 40° C. to obtain a reduced graphite oxide substrate.

(2) 0.6 g of 2,6-diaminoanthraquinone is dissolved in a DMF solution and stirred for 1 h, then reduced graphite oxide is added into the above solution and stirred continuously for 2 h, and then the ultrasonic treatment is performed for 4 h; when the mixed solution is heated to 60° C., isoamyl nitrite is added, and reaction is performed for 18 h; and reaction products are washed with ethanol and deionized water for multiple times and finally freeze dried to obtain a target product.

2. Preparation of the IT-RGO-3 composite material electrode is the same as that in embodiment 1;

3. Test of electrochemical performance: the test method is the same as that in embodiment 1; and the test result is: when the current density is 1 A g⁻¹, the specific capacitance of the electrode material can reach up to 356.7 F g⁻¹.

We claim:

1. Preparation and application of 2,6-diaminoanthraquinone bifunctional group covalently grafted graphene as a negative material of a supercapacitor, comprising the following technological steps:
(1) dispersing graphite oxide in deionized water, stirring for 1-2 h in advance, and then performing ultrasonic treatment for 2-6 h; and adding 10-15 ml of hydrazine hydrate at 80-110° C., and vacuum drying at 40-80° C. to obtain a reduced graphite oxide substrate;
(2) dissolving 2,6-diaminoanthraquinone in a DMF solution, stirring for 1-2 h, adding the reduced graphite oxide into the above solution, continuously stirring for 2-4 h, and then performing the ultrasonic treatment for 4-8 h; when the mixed solution is heated to 60-90° C., adding isoamyl nitrite, and reacting for 18-24 h; washing reaction products with ethanol and deionized water for multiple times, and finally freeze drying to obtain the 2,6-diaminoanthraquinone bifunctional group covalently grafted graphene.

2. The preparation and application of 2,6-diaminoanthraquinone bifunctional group covalently grafted graphene as the negative material of the supercapacitor according to claim 1, wherein a mass ratio of 2,6-diaminoanthraquinone to the graphite oxide is 0.1:1-0.4:2.

3. The preparation and application of 2,6-diaminoanthraquinone bifunctional group covalently grafted graphene as the negative material of the supercapacitor according to claim 1, wherein a mass ratio of 2,6-diaminoanthraquinone to the graphite oxide is 0.2:1-0.6:1.

4. An application of 2,6-diaminoanthraquinone bifunctional group covalently grafted graphene as the negative material of the supercapacitor prepared by the method of claim 1 as an electrode material of the supercapacitor.
